(19) United States
(12) Patent Application Publication
(10) Pub. No.: US 2019/0192416 A1
(43) Pub. Date: Jun. 27, 2019

(54) Formulation with a marine bio-active complex

(71) Applicants: Christopher Perez, New York, NY (US); Joseph R. Perez, Commack, NY (US)

(72) Inventors: Christopher Perez, New York, NY (US); Joseph R. Perez, Commack, NY (US)

(21) Appl. No.: 16/350,660

(22) Filed: Dec. 17, 2018

Related U.S. Application Data
(60) Provisional application No. 62/609,622, filed on Dec. 22, 2017.

(51) Int. Cl. A61K 8/9722; A61K 8/9728; A61K 8/92; A61K 8/66; A61K 8/19; A61Q 11/00

(52) U.S. Cl. CPC A61K 8/9722; A61K 8/9728; A61K 8/922; A61K 8/66; A61K 8/60; A61Q 11/00; A61K 2800/522; A61K 2800/524; A61K 2800/85; A61K 8/19

(57) ABSTRACT

The present invention provides for a formulation containing a marine bio-active complex that can be used as mouth waste, mouth rinse and toothpaste.

### RELATED APPLICATION

This application is a related to U.S. Provisional Application Ser. No. 62/609,992 filed on Dec. 22, 2017 entitled “Formulation with a Marine Bio-Active Complex”.

### BACKGROUND OF INVENTION

### Field of Invention

The present invention relates a formulation containing a marine bio-active complex. More specifically, the present invention relates to a dental formulation including mouthwashes, rinses and toothpaste. The present invention also relates to method of manufacture and applications of use.

### SUMMARY OF INVENTION

In one embodiment, the present invention provides for a formulation comprising a marine bio-active complex, and the complex comprises: cyanobacter extract, marine collagen saccharomyces, adenosine triphosphate, adenylate cyclase, at least one ferment, said ferment being selected from a group comprising of sodium, calcium, magnesium, sea salt, blue green algae, sea water, red algae and mixthres and combinations thereof.

In another embodiment, the formulation further comprises of at one carrier, said carrier is selected from a group consisting essentially of purified water, water, saline, alcohol, and mixtures and combinations thereof.

In still another embodiment, the formulation further comprises at least one humectant, said humectant is selected from a group consisting essentially of natural and synthetic humectants, glycols, alpha hydroxyl acids, glycerin, sorbitol, glycol (propylene, hexylene, butylene, caprylyl) collagen, aloe vera, honey, lactic acid, glyceryl triacetate, molasses, lithium chloride, polymeric polyois, quillaia, glycerol, maltitol, sodium hexametaphosphate, and mixtures and combinations thereof.

In yet another embodiment, the formulation further comprises at least one antimicrobial, said antimicrobial is selected from a group consisting essentially of chitosan, essential oils, lavender oils, bergamot oil, lemongrass oil, oregano oil, thyme oil, basil oil, rosemary oil, echinacea, goldenseal, tea tree oil, silver, witch hazel, rosemary extract, chamomile extract, myrrh extract, orange extract, grapefruit extract, boswellia extract, cypress extract, licorice extract, lemon oil, spearmint oil, clove oil, peppermint oil, patchouli oil, mandarin oil, cinnamon oil, eucalyptus oil, limonene, sandalwood oil, thyme oil, seabuckthorn extract, bentonite, radish root, silver, copper, cobalt, nickel, zinc, lead, zirconium, molybdenum, alloys, metals and mixtures and combinations thereof.

In still yet another embodiment, the formulation further comprises at least one sweetener, said sweetener is selected from a group consisting essentially of natural and artificial sugars, xylitol, stevia, sucralose, sorbitol, and mixtures and combinations thereof.

In a further embodiment, the formulation further comprises at least one antioxidant, said antioxidant is selected from a group consisting essentially of enzymes, superoxide dismutase (SOD), glutathione peroxidase, glutathione reductase, and catalases, vitamins, Vitamins A, C, E, folic acid, and beta-carotene, phytochemicals, carotenoids, flavonoids, allyl sulfides, polyphenols, seaweed extract, blue green algae, red algae and mixtures and combinations thereof.

In another further embodiment, the formulation further comprises at least one emulsifier, said emulsifier is selected from a group consisting essentially of lecithin, esters, silicates, oils, phosphates, polyethylene glycol, sodium tripolyphosphate, sodium lauryl phosphate, surfactants, polysorbate, sulfate castor oil, xanthan gum, magnesium aluminum silicate, bentonite and mixtures and combinations thereof.

In yet a further embodiment, the formulation further comprises at least one catalyst, said catalyst is selected from a group consisting essentially of enzyme, chemical and non-chemical catalysts, ubiquinone, adenosine triphosphate, adenylyl cyclase and mixtures and combinations thereof.

In still a further embodiment, the formulation further comprises at least one flavoring, said flavoring is selected from a group consisting essentially of natural and artificial flavors agents, spearmint oil, menthol, clove, orange oil, peppermint oil, lemon oil, patchouli oil, rosemary oil, myrrh oil, frankincense oil, chamomile oil, eucalyptus oil, mandarin oil, sandalwood oil, thyme oil, cinnamon oil, licorice oil, and mixtures and combinations thereof.

In still yet a further, the formulation further comprises preservatives and coloring.

In another embodiment, the present invention provides for a formulation comprising a marine bio-active complex, and the complex comprises: at least one extract, said extract being selected from a group comprising of blue green algae, red algae, brown algae, collagen, seaweed extract and mixtures and combinations thereof; at least one ferment, said ferment being selected from a group comprising of sodium, calcium, magnesium, and mixtures and combinations thereof; at least one saccharomyces, said saccharomyces being selected from a group comprising of marine collagen saccharomyces, and mixtures and combinations thereof; and at least one enzyme, said enzyme being selected from a group comprising adenosine triphosphate, adenylate cyclase, and mixtures and combinations thereof.

In a further embodiment, the present invention relates to a method of manufacturing a marine bio-active complex, and the method comprises admixing: at least one extract, said extract being selected from a group comprising of blue green algae, red algae, brown algae, collagen, seaweed extract and mixtures and combinations thereof; at least one ferment, said ferment being selected from a group comprising of sodium, calcium, magnesium, and mixtures and combinations thereof; at least one saccharomyces, said saccharomyces being selected from a group comprising of marine collagen saccharomyces, and mixtures and combinations thereof; and at least one enzyme, said enzyme being selected from a group comprising adenosine triphosphate, adenylate cyclase, and mixtures and combinations thereof.

In another further embodiment, the present invention provides for a method of manufacturing a formulation, said method comprises admixing: at least one carrier, at least one humectant, at least one antimicrobrial, at least one sweeter, at least one antioxidant, at least one emulsifier, at least one catalyst, and at least one flavoring.

### DETAILED DESCRIPTION OF THE INVENTION

As required, detailed embodiments of the present invention are disclosed herein; it is to be understood that the disclosed embodiments are merely exemplary of the invention that may be embodied in various forms. Therefore, specific structural and functional details disclosed herein are not to be interpreted as limits, but merely as a basis for teaching one skilled in the art to employ the present invention. The specific examples below will enable the invention to be better understood. However, they are given merely by way of guidance and do not imply any limitation.

In one embodiment, the formulation as shown in Example 1 (below) is a mouth waste or mouth rinse which is a blend of bioactive ingredients shown to reduce oral inflammation, strengthen oral immune support, and prevent plaque causing bacteria and malodor. Designed to be used daily to restore fresh breath and promote healthy oral hygiene.

### EXAMPLE 1

Purified water

Glycerin

Sucralose

Xylitol

Sodium Bicarbonate

Rosmarinus Officinalis (Rosemary) Leaf Extract

Anthemis Nobilis (Chamomile) Extract

Commiphora Myrrha (Myrrh) Extract

Citrus paridisi (grapefruit) seed extract

Thymus Vulgaris

Glycyrhiza Glabra (Licorice) Root Extract Bio-Active Marine Complex [Cyanobacter (Blue Green Algae) Extract and Marine Collagen Saccharomyces/Sodium Ferment (and) Saccharomyces/Calcium Ferment (and) Saccharomyces/Magnesium Ferment (and) Adenosine Triphosphate (and Adcnylate Cyclase]

Cupressus Sempei viieus (Cypress) Oil

Boswellia carteri (Frankincense) Oil

NatraGem

Natural Mint

Melaleuca Altemifolia (Tea Tree) Oil

Citrus Limon (Lemon) Peel Oil

Citrus aurantium dulcis (orange oil)

Menthol

In one embodiment, the formulation as shown in Example 2 (below) is a toothpaste which is a blend of bioactive ingredients shown to reduce oral inflammation, strengthen oral immune support, and prevent plaque causing bacteria and malodor.

EXAMPLE 2

DI Water

Calcium Carbonate

Xylitol

Magnesium Aluminum Silicate

Sorbitol

Glycerin

Natural & Artificial Flavor

Xanthum Gum

Sea Sat

Bentonite

Boswellia Serrate Gum Extract

Butylene Glycol

Caprylyl Glycol

Chamomilla Recutita (Marticaria) Flower Extract

Cupressus Smepervirens (Cypress) Leaf Extract

Glycyrhiza Glabra (Licorice) Root Extract

Hippohae Rhamnodies (Seabuckthorn) Fruit Extract

Kappaphycus Alvarezil Extract

Leuconotstoc/Radish Root Ferment Filtrate

L-Menthol

Maris Aqua (Sea Water)

Phenoxyethanol

Potassium Sorbate

Propylene Glycol

Pseudoalteromonas Ferment Extract

Rosmarinus Officinalis (Rosemary) Leaf Extract

Silver

Sodium Benzoate

Sodium Lauroyl Sarcosinatc

Thymus Vulgaris (Thyme) Leaf Extract

The present invention is not limited to the above Examples and Tables. Numerous modifications and variations of the present invention are possible in light of the above teachings. It is therefore to be understood that within the scope of the attendant claims attached hereto, this invention may be practiced other than as specifically disclosed herein.

What is claimed is:

1. A formulation comprising a marine bio-active complex, said complex comprises: cyanobacter extract, marine collagen saccharomyces, adenosine triphosphate, adenylate cyclase, at least one ferment, said ferment being selected from a group comprising of sodium, calcium, magnesium, sea salt, blue green algae, sea water, red algae and mixtures and combinations thereof.

2. The formulation of claim 1 further comprising of at one carrier, said carrier is selected from a group consisting essentially of purified water, water, saline, alcohol, and mixtures and combinations thereof.

3. The formulation of claim 1 further comprising at least one humectant, said humectant is selected from a group consisting essentially of natural and synthetic humectants, glycols, alpha hydroxyl acids, glycerin, sorbitol, glycol (propylene, hexylene, butylene, caprylyl) collagen, aloe vera, honey, lactic acid, glyceryl triacetate, molasses, lithium chloride, polymeric polyois, quillaia, glycerol, maltitol, sodium hexametaphosphate, and mixtures and combinations thereof.

4. The formulation of claim 1 further comprising at least one antimicrobial, said antimicrobial is selected from a group consisting essentially of chitosan, essential oils, lavender oils, bergamot oil, lemongrass oil, oregano oil, thyme oil, basil oil, rosemary oil, echinacea, goldenseal, tea tree oil, silver, witch hazel, rosemary extract, chamomile extract, myrrh extract, orange extract, grapefruit extract, boswellia extract, cypress extract, licorice extract, lemon oil, spearmint oil, clove oil, peppermint oil, patchouli oil, mandarin oil, cinnamon oil, eucalyptus oil, limonene, sandalwood oil, thyme oil, seabuckthorn extract, bentonite, radish root, silver, copper, cobalt, nickel, zinc, lead, zirconium, molybdenum, alloys, metals and mixtures and combinations thereof.

5. The formulation of claim 1 further comprising at least one sweetener, said sweetener is selected from a group consisting essentially of natural and artificial sugars, xylitol, stevia, sucralose, sorbitol, and mixtures and combinations thereof.

6. The formulation of claim 1 further comprising at least one antioxidant, said antioxidant is selected from a group consisting essentially of enzymes, superoxide dismutase (SOD), glutathione peroxidase, glutathione reductase, and catalases, vitamins, Vitamins A, C, E, folic acid, and beta-carotene, phytochemicals, carotenoids, flavonoids, allyl sulfides, polyphenols, seaweed extract, blue green algae, red algae and mixtures and combinations thereof.

7. The formulation of claim 1 further comprising at least one emulsifier, said emulsifier is selected from a group consisting essentially of lecithin, esters, silicates, oils, phosphates, polyethylene glycol, sodium tripolyphosphate, sodium lauryl phosphate, surfactants, polysorbate, sulfate castor oil, xanthan gum, magnesium aluminum silicate, bentonite and mixtures and combinations thereof.

8. The formulation of claim 1 further comprising at least one catalyst, said catalyst is selected from a group consisting essentially of enzyme, chemical and non-chemical catalysts, ubiquinone, adenosine triphosphate, adenylyl cyclase and mixtures and combinations thereof.

9. The formulation of claim 1 further comprising at least one flavoring, said flavoring is selected from a group consisting essentially of natural and artificial flavors agents, spearmint oil, menthol, clove, orange oil, peppermint oil, lemon oil, patchouli oil, rosemary oil, myrrh oil, frankincense oil, chamomile oil, eucalyptus oil, mandarin oil, sandalwood oil, thyme oil, cinnamon oil, licorice oil, and mixtures and combinations thereof.

10. The formulation of claim 1 further comprising preservatives and coloring.

11. A formulation comprising a marine bio-active complex, said complex comprises:
at least one extract, said extract being selected from a group comprising of blue green algae, red algae, brown algae, collagen, seaweed extract and mixtures and combinations thereof;
at least one ferment, said ferment being selected from a group comprising of sodium, calcium, magnesium, and mixtures and combinations thereof;
at least one saccharomyces, said saccharomyces being selected from a group comprising of marine collagen saccharomyces, and mixtures and combinations thereof; and
at least one enzyme, said enzyme being selected from a group comprising adenosine triphosphate, adenylate cyclase, and mixtures and combinations thereof.

12. The formulation of claim 12 further comprising of at least one carrier and at least one sweetener, said carrier is selected from a group consisting essentially of purified water, water, saline, alcohol, and mixtures and combinations thereof, said sweetener is selected from a group consisting essentially of natural and artificial sugars, xylitol, stevia, sucralose, sorbitol, and mixtures and combinations thereof.

13. The formulation of claim 12 further comprising at least one humectant, said humectant is selected from a group consisting essentially of natural and synthetic humectants, glycols, alpha hydroxyl acids, glycerin, sorbitol, glycol (propylene, hexylene, butylene, caprylyl) collagen, aloe vera, honey, lactic acid, glyceryl triacetate, molasses, lithium chloride, polymeric polyois, quillaia, glycerol, maltitol, sodium hexametaphosphate, and mixtures and combinations thereof.

14. The formulation of claim 12 further comprising at least one antimicrobrial, said antimicrobial is selected from a group consisting essentially of chitosan, essential oils, lavender oils, bergamot oil, lemongrass oil, oregano oil, thyme oil, basil oil, rosemary oil, echinacea, goldenseal, tea tree oil, silver, witch hazel, rosemary extract, chamomile extract, myrrh extract, orange extract, grapefruit extract, boswellia extract, cypress extract, licorice extract, lemon oil, spearmint oil, clove oil, peppermint oil, patchouli oil, mandarin oil, cinnamon oil, eucalyptus oil, limonene, sandalwood oil, thyme oil, seabuckthorn extract, bentonite, radish root and mixtures and combinations thereof.

15. The formulation of claim 12 further comprising at least one antioxidant, said antioxidant is selected from a group consisting essentially of enzymes, superoxide dismutase (SOD), glutathione peroxidase, glutathione reductase, and catalases, vitamins, Vitamins A, C, E, folic acid, and beta-carotene, phytochemicals, carotenoids, flavonoids, allyl sulfides, polyphenols, seaweed extract, blue green algae, red algae and mixtures and combinations thereof.

16. The formulation of claim 12 further comprising at least one emulsifier, said emulsifier is selected from a group consisting essentially of lecithin, esters, silicates, oils, phosphates, polyethylene glycol, sodium tripolyphosphate, sodium lauryl phosphate, surfactants, polysorbate, Sulfate castor oil, xanthan gum, magnesium aluminum silcate, bentonite and mixtures and combinations thereof.

17. The formulation of claim 12 further comprising at least one catalyst, said catalyst is selected from a group consisting essentially of enzyme, chemical and non-chemical catalysts, ubiquinone, adenosine triphosphate, adenylyl cyclase and mixtures and combinations thereof.

18. The formulation of claim 12 further comprising at least one flavoring, at least one preservatives and at least one coloring, said flavoring is selected from a group consisting essentially of natural and artificial flavors agents, spearmint oil, menthol, clove, orange oil, peppermint oil, lemon oil, patchouli oil, rosemary oil, myrrh oil, frankincense oil, chamomile oil, eucalyptus oil, mandarin oil, sandalwood oil, thyme oil, cinnamon oil, licorice oil, and mixtures and combinations thereof.

19. A method of manufacturing a marine bio-active complex, said method comprises admixing: at least one extract, said extract being selected from a group comprising of blue green algae, red algae, brown algae, collagen, seaweed extract and mixtures and combinations thereof; at least one ferment, said ferment being selected from a group comprising of sodium, calcium, magnesium, and mixtures and combinations thereof; at least one saccharomyces, said saccharomyces being selected from a group comprising of marine collagen saccharomyces, and mixtures and combinations thereof; and at least one enzyme, said enzyme being selected from a group comprising adenosine triphosphate, adenylate cyclase, and mixtures and combinations thereof.

20. A method of manufacturing a formulation, said method comprises admixing: at least one carrier, at least one humectant, at least one antimicrobrial, at least one sweeter, at least one antioxidant, at least one emulsifier, at least one catalyst, and at least one flavoring.
